(19) United States
(12) Patent Application Publication
(10) Pub. No.: US 2025/0107854 A1
(43) Pub. Date: Apr. 3, 2025

(54) BRONCHOSCOPE GRAPHICAL USER INTERFACE WITH IMPROVED NAVIGATION

(71) Applicant: CANON U.S.A., INC., MELVILLE, NY (US)

(72) Inventors: Charles George Hwang, Wellesley, MA (US); Takahisa Kato, Brookline, MA (US)

(21) Appl. No.: 18/730,714

(22) PCT Filed: Feb. 6, 2023
(86) PCT No.: PCT/US2023/012404

§ 371 Date: Jul. 19, 2024

Related U.S. Application Data
(60) Provisional application No. 63/307,878, filed on Feb. 8, 2022.

(51) Int. Cl. A61B 34/20; A61B 1/00; A61B 1/01; A61B 1/267

(52) U.S. Cl. CPC A61B 34/20; A61B 1/0005; A61B 1/00193; A61B 1/01; A61B 1/267

(57) ABSTRACT

There is provided a navigation system, a medical system, a method of use, and media for using in navigation of a bendable medical device. The system and method comprises a display device and a controller, where the controller is configured to display on the display device an image of a biological lumen imaged from the distal end of a bendable medical device; and a representation of an airway structure. The representation of the airway structure includes: a navigation path through at least a portion of the airways; and a guidance reference plane located perpendicular to navigation path and located at an insertion depth of the distal end of the bendable medical device.

### CROSS-REFERENCE TO RELATED APPLICATIONS

The present application claims priority to U.S. provisional application 63/307,878 filed 8 Feb. 2022. The disclosures of the above-listed provisional application is hereby incorporated by reference in its entirety for all purposes. Priority benefit is claimed under 35 U.S.C. § 119(e).

### BACKGROUND INFORMATION

### Field of Disclosure

The present disclosure relates generally to systems and methods for medical applications. More particularly, the subject disclosure is directed to a system using an articulated medical device and the display of information from the medical device, wherein the medical device is capable of maneuvering within a patient.

### Description of Related Art

Bendable medical devices such as endoscopic surgical devices and catheters are well known and continue to gain acceptance in the medical field. The bendable medical device generally includes a flexible body commonly referred to as a sleeves or sheaths. One or more tool channels extend along (typically inside) the flexible body to allow access to a target site located at a distal end of the body.

The medical device is intended to provide flexible access within a patient, with at least one curve or more leading to the intended target, while retaining torsional and longitudinal rigidity so that a clinical user can control the tool located at the distal end of the medical device by maneuvering the proximal end of the device.

The medical device may be implemented via a system, where the system includes both hardware and software that when used together allow the user to guide and observe the movement of the medical device through passageways within a patient. By way of example, United States Patent Publication number 2019/0105468, describes such a system for implementing an articulated medical device having a hollow cavity, where the device is capable of maneuvering within a patient, and allowing a medical tool to be guided through the hollow cavity for medical procedures, including endoscopes, cameras, and catheters.

To aid in the navigation of the medical device towards a target site, a physician may use pre-operative and/or intra-operative imaging techniques, such as Computed Tomography (CT), Magnetic Resonance Imaging (MRI), ultrasound (US), or other similar techniques to provide a ‘roadmap’ for navigation the surgical tools through or around internal structures and organs of a patient. For navigation through the lung, while the lung structure adventitiously provides multiple branching pathways in order to choose the best route to a target, there is the added complication that, with the continued branching of the airways, it can sometimes be difficult to determine which airway the surgical tool is in relative to the target. An image of the entire lung structure may be provided to aid navigation. However, in order for the user to steer the catheter to the target, the user needs to understand the position of the medical device relative to the entire lung structure, the progress along a planned navigation path, and the possible airways available for orientation.

The difficulty is that current state-of-the-art “road maps” fall into one of two categories: a current view model (see, for example, U.S. Pat. No. 9,727,963) that provides only the current airways ‘viewed’ by the medical device and a 2D representation model (see, for example, U.S. Pat. Pub. 2020/0054399), which does not provide airway options or structure details. Similarly deficient teachings are provide in U.S. Pat. Nos. 10,617,324; 8,337,397; U.S. Pat. Pub. 2020/0054399; U.S. Pat. Pub. 2019/0254649 and WO 2018/00586.

Thus, there is need for an improved display and system to aid in the navigation and use of bendable medical devices in the lung. The improved display will aid in visualization of the location in the airway structure the tip of the bendable medical device; visualization of where along the navigation path the tip is, and a virtual endoscope view to see the options available and to orient the live endoscope view.

### SUMMARY OF EXEMPLARY EMBODIMENTS

According to at least one embodiment of the invention, there is provided a navigation system comprising: a display device; and a controller configured to display on the display device: an image of a biological lumen imaged from the distal end of a bendable medical device; and a representation of an airway structure. The representation of an airway structure, which may be, for example, a segmented model of the airways obtained from a CT image, also has included on the model image: a navigation path through at least a portion of the airway structure; and a guidance reference plane oriented perpendicular to the navigation path and located at an insertion depth of the distal end of the bendable medical device in the biological lumen. The centerline, the target site, a ring or other feature depicting the location of the endoscope tip may also be include don the representation of the airway structure.

The guidance reference plane is optionally updated as the bendable medical device is moved through the airway structure. The controller may be configured to display a guidance virtual endoscope view having an image center at the centerline on the guidance reference plane and a view orientation of the distal direction along the orientation of the guidance reference plane. Alternatively or additionally, the controller is configured to display a second virtual endoscope view having an image center at the distal end of the bendable medical device.

The controller also may be configured to display at least one of: a navigation modality, distance from the bendable medical device to target site; insertion depth of the bendable medical device; information as to the position of a marker displayed on the airway structure; and warning(s) that the bendable medical device is reaching a threshold limit of force or bending angle.

The navigation system as described herein may also display one or more markers indicating one or more bifurcation points of the airway structure. These can be selected and the guidance reference plane can be moved to the selected bifurcation point of the airway structure to become an overview guidance reference plane.

Methods, and computer-readable storage medium for performing the steps of the method are also provided. For example, a method including the steps of acquiring an image of a biological lumen from the distal end of the bendable medical device; obtaining a representation of an airway structure; obtaining a target site; generating a centerline of at least a portion of the airway structures; generating a navigation path through at least a portion of the airway structure; displaying, on a display device, the representation of an airway structure, wherein the a representation of an airway structure includes: the navigation path, and a guidance reference plane located perpendicular to navigation path and located at an insertion depth of the distal end of the bendable medical device is provided.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 illustrates an exemplary embodiment of a robot-assisted endoscope system 1000 in medical environment, such as an operating room;

FIG. 2 illustrates an example embodiment of a system to allow a user to guide and observe the movement of a medical device within a patient.

FIG. 3 illustrates an example embodiment of a steerable medical system 1000 represented in functional block diagram.

FIG. 4 illustrates a lung with a pathway for endoscope insertion.

FIG. 5 illustrates an endoscope view of an airway structure.

FIGS. 6(A) and 6(B) provide exemplary displays showing a virtual endoscope view, a live endoscope view, and a representation of a lung.

FIGS. 7(A) and 7(C) provides other exemplary display showing a virtual endoscope view, a live endoscope view, and a representation of a lung.

FIGS. 7(B) and 7(D) provide the actual catheter position in the lung for the images of FIG. 7(A) and FIG. 7(C), respectively.

FIG. 8(A), FIG. 8(B), and FIG. 8(C) provides other exemplary display showing a virtual endoscope view, a live endoscope view, and a representation of a lung.

FIG. 9 provides an exemplary display showing a virtual endoscope view, a live endoscope view, and a representation of a lung.

FIG. 10 provides an exemplary display showing a virtual endoscope view, a live endoscope view, and a representation of a lung.

Throughout the Figures, the same reference numerals and characters, unless otherwise stated, are used to denote like features, elements, components or portions of the illustrated embodiments. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

Unless specifically stated otherwise, as apparent from the following disclosure, it is understood that, throughout the disclosure, discussions using terms such as “processing,” “computing,” “calculating,” “determining,” “displaying,” or the like, refer to the actions and processes of a processor such as a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computational or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

As used herein the term “bendable medical device” generally refers to a flexible and thin tubular instrument made of medical grade material designed to be inserted through a narrow opening into a bodily lumen (e.g., a vessel or bronchi) to perform a broad range of medical functions. A catheter can be a bendable medical device. The more specific term “optical catheter” refers to a bendable medical device comprising an elongated bundle of one or more flexible light conducting fibers disposed inside a protective sheath made of medical grade polymer material and having an optical imaging function. A particular example of an optical catheter is a fiber optic catheter which comprises a flexible sheath, a coil, and an optical probe or imaging core contained within the coil. In some applications a catheter may include a “guide catheter” which functions similarly to a sheath. The bendable medical device may be configured for use with one or more tools. For example, a camera may be inserted into the bendable medical device, or a camera or other optical probe may be an integral part of the device. Biopsy instruments may also be used with the bendable medical device.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth and lung), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes. Embodiments of the present disclosure can be applicable to one or more of the foregoing endoscopes.

The present disclosure generally relates to medical devices, and it exemplifies embodiments of an optical probe which may be applicable to an imaging apparatus (e.g., an endoscope. The embodiments of the optical probe and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

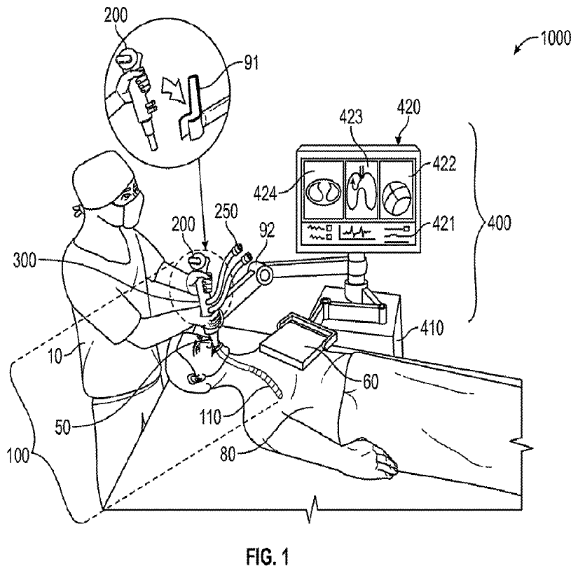

An exemplary configuration of a medical system 1000 such as a robot-assisted endoscope system is described with reference to FIG. 1. This figure illustrates an example representation of a medical environment such as an operating room where a robot-assisted endoscope system 1000 can be practiced. The robot-assisted endoscope system 1000 may include a steerable instrument 100 (a steerable medical device) operable by a user 10 (e.g., a physician) to perform an endoscopy procedure on a patient 80. The robot-assisted endoscope system 1000 may include a computer system 400 operatively attached to the steerable instrument 100 via a robotic platform 90. The computer system 400 (e.g., a system console) includes a processor or central processing unit (CPU) 410 and a display screen 420 such as a liquid crystal display (LCD), OLED or QLED display. A storage memory 411 (ROM and RAM memory), a system interface 412 (FPGA card), and a user interface 413 (e.g. mouse and keyboard) are operatively connected to the processor or CPU 410 and to the display screen 420.

The steerable instrument 100 includes a handle 200 and a bendable medical device (e.g., a steerable sheath) 110, which are removably connected to each other by a connector assembly 50. The handle 200 includes an actuator system 300 which receives electronic commands from computer system 400 to mechanically actuate the bendable medical device 110. The handle 200 is configured to be detachably mounted on the robotic platform 90. The robotic platform 90 includes a robotic arm 92 and a stage 91 for robotically guiding the bendable medical device 110 towards a target site 82 within the subject or patient 80. When the handle 200 is not necessarily mounted on the robotic platform 90, the handle 200 can be operated manually by the user 10 to control the bendable medical device 110. For treating or examining the patient 80, the steerable instrument 100 may include one or more access ports 250 arranged on or around the handle 200. Access ports 250 can be used for inserting end effectors or for passing fluids to/from the patient. An electromagnetic (EM) field generator 60 interacts with one or more EM sensors 190 arranged on the steerable sheath 110 for tracking the position, shape, and/or orientation of the steerable sheath 110 while being inserted through a bodily lumen 81 towards a target site 82 within the patient 80. The medical device 110 may include a tool channel for a biopsy or other interventional tool. The clinical user can insert and retreat the medical device 110 to perform, for example, a biopsy in the airways of the patient.

During an endoscope procedure, the system processor or CPU 410 of computer system 400 is configured to perform operations based on computer-executable code pre-stored in the system's memory 411. The display screen 420 may include a graphical user interface (GUI) configured to display one or more of patient information in an information window 421, an endoscope live-image 422, an intra-operative image 423 (e.g., fluoroscopy), and a pre-operative image 424 (e.g., a slice image) of the patient 80.

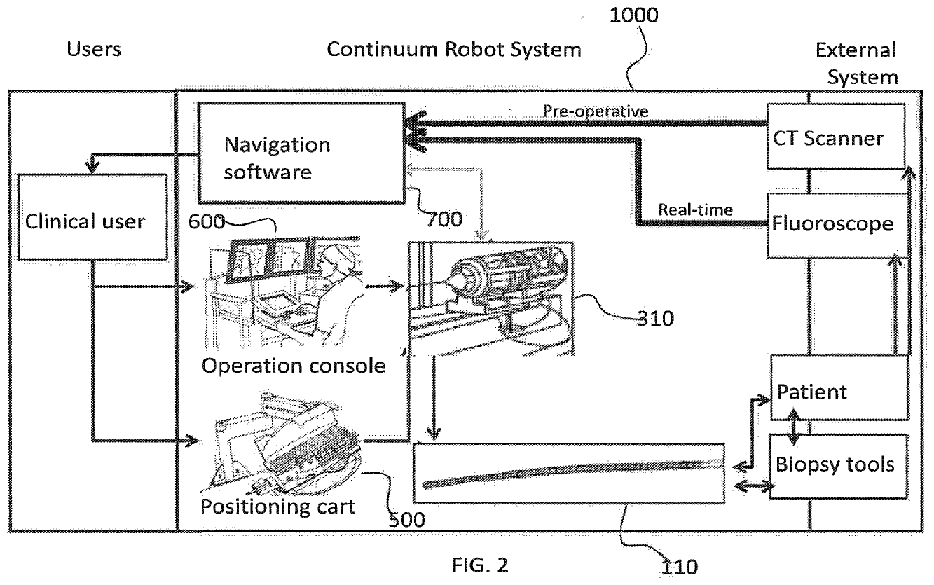

FIG. 2 illustrates an example embodiment of a medical system 1000. The medical system 1000 (also referred herein as a continuum robot system) comprises a driving unit 310, a bendable medical device 110 (or sheath), a positioning cart 500, an operation console 600, and navigation software 700. The system 100 also interacts with clinical users and external systems (e.g., a computerized tomography (CT) scanner and/or magnetic resonance imaging (MRI) scanner, a fluoroscope, a patient, biopsy tools).

The navigation software 700 and the driving unit 310 are communicatively coupled via a bus, which transmits data between them. Moreover, the navigation software 700 may be coupled to and communicates with a CT scanner or MRI scanner, a fluoroscope, and an image server (not in FIG. 2), which are external of the medical system 1000. The image server may be, for example, a DISCOM server that is coupled to a medical imaging device, such as a CT scanner, a MRI scanner, and a fluoroscope. The navigation software 700 processes data provided by the driving unit 310, data provided by images stored on the image server, images from the CT scanner/MRI scanner, and images from the fluoroscope in order to display images on a display device.

The images from the CT scanner/MRI scanner are pre-operatively provided to the navigation software 700. With the navigation software 700, a clinical user can create an anatomical computer model from the images. In some embodiments, the anatomy is a biological lumen such as the lung airway. From the chest images of the CT scanner/MRI scanner, the clinical user can segment the lung airways for clinical use. Thus, a lung-airway map may be created from this data and this lung-airway map may be used to create a planned path. With or without the path, the data can also be used to guide or inform treatments, such as a biopsy using the bendable medical device 110 inserted into the biological lumen. In other embodiments, images from, for example, inter-operative fluoroscopy may be used by the navigation software 700 to create (or add to) an anatomical computer model of the biological lumen

The driving unit 310 comprises actuators and a control circuitry. The control circuitry is communicatively-coupled with the operation console 600. Also, the driving unit 310 is connected to the bendable medical device 110 so that the actuators in the driving unit 310 operate the medical device 110. Therefore, a clinical user can control the medical device 110 via the driving unit 2. The driving unit 310 is also physically connected to a positioning cart 500. The positioning cart 500 may include one or more positioning arm(s) and a translational stage, and the positioning cart 500 locates the driving unit 310 and the medical device 110 in the intended position against a patient.

The operation console 600, optimally includes one or more displays as well as an input device such as a mouse, joystick, touchscreen, voice activation, or similar.

The medical device may comprise a camera at the distal tip of the medical device (i.e., a ‘chip-on-tip design). Alternatively, the medical device will comprise an imaging means for generating an image of the region at the distal tip. For example, the image may be generated via traditional CCD endoscope, a borescope, a fiberscope, or by spectrally encoded endoscopy (see, for example, U.S. Pat. Nos. 7,551,293; 9,295,391; 10,288,868; and 10,401,610). The medical device will form an image at the distal end of the tip. This image can be used for navigation of the flexible medical device. This image of the interior of the biological lumen (e.g., a lung) or other hollow organ (e.g., renal pelvis) can be combined with the CT, MRI, fluoroscope or other image taken of the area of interest to aid in guidance of the medical device towards a target site.

The medical device 110 may include a tool channel for a biopsy or other interventional tool. Thus, the medical device 110 can guide the biopsy tool to the lesion of the patient. The clinical user can take a biopsy sample from the lesion with the biopsy tool.

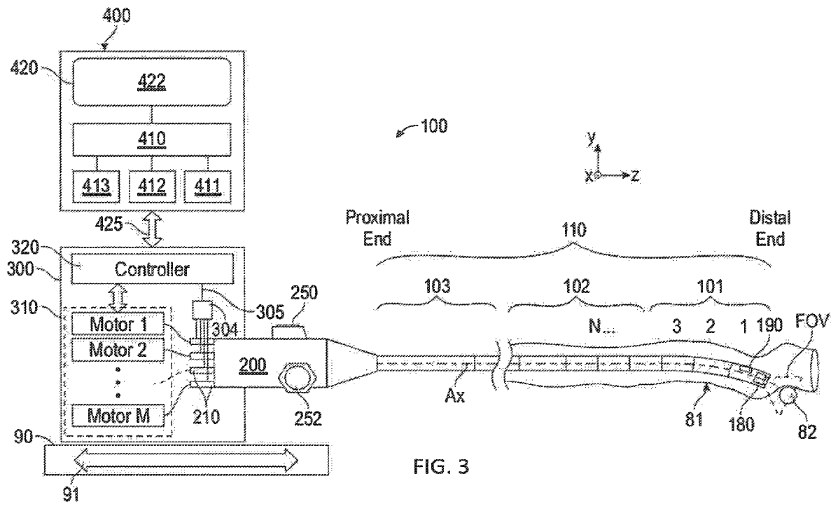

FIG. 3 illustrates a general structure of the steerable medical system 1000 from FIG. 1 in functional block diagram without the user and/or patient. The medical system 1000 includes a handle 200 and a bendable medical device 110, which are removably connected to each other by a connector assembly 50. The handle 200 includes an actuator system 300 that is part of the driving unit 310 and which receives electronic commands from computer system 400 to mechanically actuate the bendable medical device 110. The handle 200 is configured to be detachably mounted on the robotic platform 90, which may be part of the positioning cart 500. The robotic platform 90 includes a robotic arm 92 and a stage 91 for robotically guiding the bendable medical device 110 towards a target site 82 within the subject or patient 80. When the handle 200 is not mounted on the robotic platform 90, the handle 200 can be operated manually by the user 10 to control the bendable medical device 110. For treating or examining the patient 80, the steerable medical system 1000 may include one or more access ports 250 arranged on or around the handle 200. Access ports 250 can be used for inserting end effectors or for passing fluids to/from the patient. An electromagnetic (EM) field generator 60 interacts with one or more EM sensors 190 arranged on the bendable medical device 110 for tracking the position, shape, and/or orientation of the bendable medical device 110 while being inserted through a bodily lumen 81 towards a target site 82 within the patient 80.

The steerable medical system 1000 includes a computer system 400 (e.g. a system console), a robotic actuator system 300, and a steerable medical system 100 which is connected to the actuator system 300 via a handle 200. The steerable medical system 100 includes bendable medical device 110 (also described as a steerable sheath) comprised of a proximal section 103, a middle section 102, and a distal section 101 arranged in this order along a longitudinal axis (Ax). The proximal section 103 is a non-steerable section and serves to connect the steerable section to handle 200 and the actuation system. The middle section 102 and the distal section 101 constitute a steerable section of the bendable medical device and are configured to be inserted into a bodily lumen 81 of a patient 80. The steerable distal section 101 (and middle section 102) are divided into multiple bending segments 1, 2, 3 . . . N which are configured to be bent, curved, twisted, and/or rotated when advancing the bendable medical device through intraluminal tortuous paths of a bodily lumen. Each bending segment includes at least one ring-shaped component. By convention, the steerable medical system 100 operates in a three-dimensional (3D) space defined by a 3D coordinate system of x, y, z Cartesian coordinates. The bendable medical device 110 defines at least one tool channel 105 which extends from the proximal end to the distal end along the longitudinal axis Ax. The bendable medical device 110 may include one or more position and/or orientation sensors 190 arranged on the wall the catheter sheath, and may include a removable imaging device 180, such as a fiber camera or a miniature electronic CMOS sensor arranged in the tool channel 105. The imaging device 180 is arranged such that its imaging plane is in the x-y plane, and the longitudinal axis Ax of the bendable medical device 110 extends along the z-axis of the coordinate system.

An example of a bendable medical device 110 and a method of using the medical device via the medical system 100 is described in United States Pat. Pub. No. 2019/0105468, which is incorporated by reference herein in its entirety. Other examples of bendable medical devices and methods of using the medical device via the medical system are disclosed in United States Pat. Pub. Nos. 2018/0243900; 2018/0311006; 2019/0105468; 2019/0015978; and 2019/0105468; and PCT Pub. Nos. WO2018/204202; WO/2020/086749; and WO/2020/092096, all of which are incorporated by reference herein in their entirety.

For inserting an endoscope into a biological lumen 81 such as an airway of a patient 80, the tip (distal end) of the bendable medical device 110 is advanced (navigated) along a center line of the lumen. In this case, an imaging device 180 (e.g., a miniature camera) can be arranged in the tool channel 105 to provide a live-view image of the lumen 81 taken directly from the instrument's field of view (FOV). However, in some embodiments, the bendable medical device 110 may not allow for the arrangement of a camera within the tool channel. In this case, navigation may be provided by intra-procedural guided imaging based on position and/or orientation provided by the one or more sensors 190 arranged along the sheath. In any case, in order to reach a desired target site 82, the bendable medical device 110 must bend, twist and/or rotate in different directions such that the distal section of the bendable medical device continuously changes shape and direction until it reaches an optimal location aligned with target site 82 such as a tumor.

The bending, twisting, and/or rotation (steering) of bendable medical device 110 is controlled by a system comprised of the handle 200, the actuator system 300 and/or the computer system 400. The actuator system 300 includes a micro-controller 320 and an actuator unit 310 which are operatively connected to the computer system 400 via a network connection 425. The computer system 400 includes suitable software, firmware, and peripheral hardware operated by the processor or CPU 410. The computer system 400, the actuator system 300, and the handle 200 are operably connected to each other by the network connection 425 (e.g., a cable bundle or wireless link). In addition, the computer system 400, the actuator system 300 and the handle 200 are operatively connected to each other by the robot platform 90, which may include one or more robotic arms 92 and translation stage 91, which is also incorporated in the driving unit 310. In some embodiments, the actuator system 300 may include or be connected to a handheld controller, such as a gamepad controller or a portable computing device like a smart phone or a tablet. Among other functions, the computer system 400 and actuator system 300 can provide a surgeon or other operator with a graphical user interface (GUI) and patient information shown in the display screen 420 to operate the steerable medical system 100 according to its application.

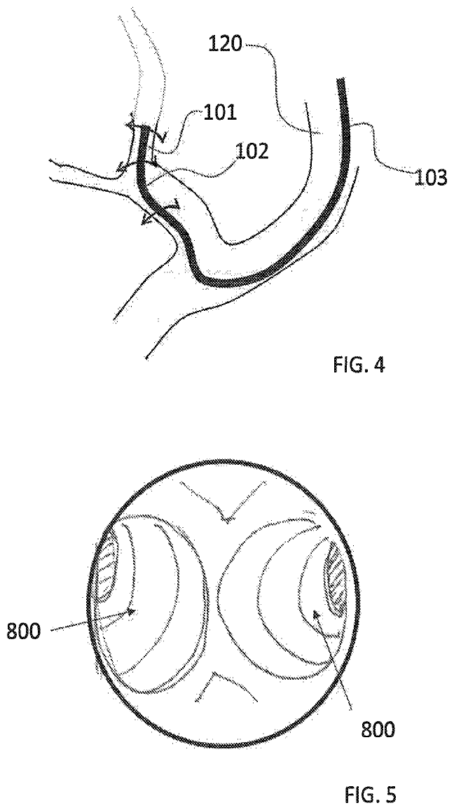

FIG. 4 illustrates the bendable medical device having three sections (101, 102, and 103), where the tip 104 of the bendable medical device 110 is the distal-most portion of the distal section 101. The bendable medical device is situation in a body lumen, which can be the lung 120. As indicated by the lung 120, at each bifurcation, the airway may become smaller such that the bendable medical device can no longer fit into the airway.

FIG. 5 shows an example endoscope view of airway structures 800 of a patient. This is a general image and has no information as to how the bendable medical device will fit or move through the airway. This view is what the clinical user can use to navigate through the lung. This view may be combined with CT or MRI imaging or fluoroscopy imaging of the lung to aid in the navigation for a medical procedure.

Thus, to achieve the goals in the previous section, a medical system such as robot-assisted endoscope system as described here provides a guidance virtual bronchoscope reference plane, or guidance reference plane, which is displayed in a view with either real-time bronchoscope view or conventional virtual bronchoscope view. The guidance virtual bronchoscope reference plane is a sectional plane that is based on the current tip position of the bendable medical device 110 and orientation at the distal end of the bendable medical device. The guidance reference plane is a cross-section view centered in the airway that makes all the path options visible instead of displaying non-cross sectional views which obscure structures and make visualization and/or navigation difficult.

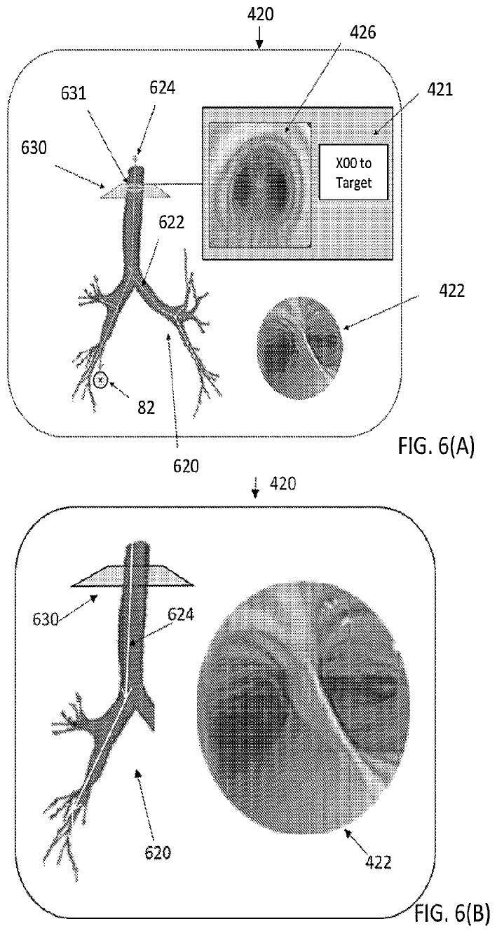

FIG. 6(A) provides a display screen 420 having a representation of the airway structure 620 and a live endoscopic view 422 from the imaging device 180. The representation of the airway structure 620 may be an anatomically correct representation of a patient lung. In some embodiments, it is the complete, anatomically correct lung. In other embodiments, it comprise at least the portion of the lung between the bendable medical device and the target site or positions as well as nearby bifurcations. The representation of the airway structure 620 may be a segmented model of the airway constructed, for example, from a CT image. The segmentation may be manual, semi-automatic, or automate and may involve machine learning algorithms. See, for example, Garcia-Ucede et al., Automatic airway segmentation from computed tomography using robust and efficient 3-D convolutional neural networks (Sci Rep 11, 16001 (2021)).

A centerline of the airway 622 is calculated by the robotic catheter system 1000. A navigation path 624 is created, which follows the centerline 622 from the trachea to the target(s) 82 or a portion thereof. As shown, the centerline 622 of each of the airways is calculated from the airway structure 620. The centerline 622 is then displayed for at least a portion of the airway structure. Based on information from the user about the location of a target, a navigation path 624 is plotted from the trachea or other point in the airway to the target 82. The navigation path 624 can be defined as a portion of the group of the centerlines in the airway that are the centerlines that define a pathway a start position to the target site 82 or to a position in the airway that is proximal to the target site in instances where the target site is not in the airway. The navigation path 624 may be defined as the various centerlines or it may have undergone a smoothing or other operation(s).

A guidance virtual bronchoscope reference plane 630 is generated and displayed. This view is perpendicular to the navigation path 624, creating a cross sectional view, centered on the center of the airway 622. The guidance reference plane 630 is a sectional plane shows the position where the cross section is taken. In this embodiment, a ring 631 indicating the position of the distal tip of the bendable medical device 110 is shown on the guidance reference plane 630. This ring 631 represents the location of the live endoscope view.

The guidance reference plane 630 is shown as a semi-transparent rectangle on the airway structure 620. Other embodiments can provide other visualizations. For example, a circle or oval can be used instead of a square. The guidance reference plane 630 could also be shown with a thickness to aid in visualization. One side of the plane could be distinguished (e.g., by color or shape) to indicate the top of the bendable medical device as defined in relation to the handle 200.

The target site 82 is the position or location in the anatomy where the user (e.g., a clinical user) intends to interact, such as to take a biopsy, perform surgery, deliver therapeutics, etc. This target site is shown on the representation of the airway structure. The location of the target site may be obtained through, for example, having the user indicate the location on the display, or it may have been annotated in the pre-procedure image and the location is taken from this information. Alternatively, the target site may be determined through the use of an algorithm trained to find tumors or other points of interest. In some embodiments, there will be multiple target sites during a procedure, and the clinical user would, for example, determine which target site should be accessed first.

Information from these calculations and/or other information, such as the distance from the bendable medical device to target site; the insertion depth of the bendable medical device; information as to the position of a marker displayed on the airway structure may be shown in an information window 421 showing patient information. Other information that may be shown include the navigation modality or modalities used, such as a label indicating that the display is showing an endoscope view, CT images, computer generated paths, etc.

The display 420 may also include a guidance virtual endoscope view 426. The guidance virtual endoscope view 426 is a virtual endoscope view that is from the point of view of the center of the guidance reference plane 630. The view orientation is the normal vector of the guidance reference plane 630 to the distal side of the steerable medical device 110. The guidance virtual endoscope view 426 has advantage to provide a good perspective of the next airways to the user when it is used with the display symbol of the guidance reference plan 630 and the airway structure 620. Unlike the normal endoscope view showing a live image 422 from the catheter tip 120, the guidance virtual endoscope view 426 always capture the airway anatomy from the center of the airway at the insertion depth position even when the catheter tip 120 orients to suboptimal direction to understand the next airway. For example, if the catheter tip 102 directed to the airway wall, the user cannot check any of the next airways expect for the wall. However, the guidance virtual endoscope view 426 would provide the view of the next airways from the current insertion depth position and help the user to operate the catheter tip 102 to optimal direction.

This information window 421 may also or alternatively provide a warning that the bendable medical device is reaching a threshold limit of force or bending angle such as where damage or system malfunction could occur if further force or increased angle is applied. This warning may be, for example, a text box indicating the warning, a change in color on the display of the region where the damage or system malfunction could occur, etc. The threshold limit of force or a limit to the bending angle may be set by the manufacturer of the bendable medical device or it may be set by the user and could be dependent on the patient or procedure. In some embodiments, the controller may initiate a corrective action such as retracting the bendable medical device, or relax one or more tendon/wire in the bendable medical device.

FIG. 6(B) shows an embodiment having a display screen that is simpler in design than the display screen of FIG. 6(A). This display screen 420 depicts a representation of the airway structure 620 where the portion of the airway away from the target site is not shown. The live endoscopic view 422 is also shown. With less of the airway and fewer views being shown, both the structure and view can be enlarged compared to other embodiments. The representation of the airway structure 620 includes both the navigation path 624 and the guidance virtual reference plane. In this embodiment, the centerline 622 and target site 82 are not shown since they may significantly overlap the navigation path 624 and are not needed for navigation. Each of these elements may be included or excluded based on preference. Additionally, it is contemplated that only a portion of the centerline 622 and/or navigation path 624 are shown on the representation of the airway structure 620. Similarly, the live endoscopic view 422 is shown. Alternatively, the guidance virtual endoscopic view 426 may be shown in place of the live view.

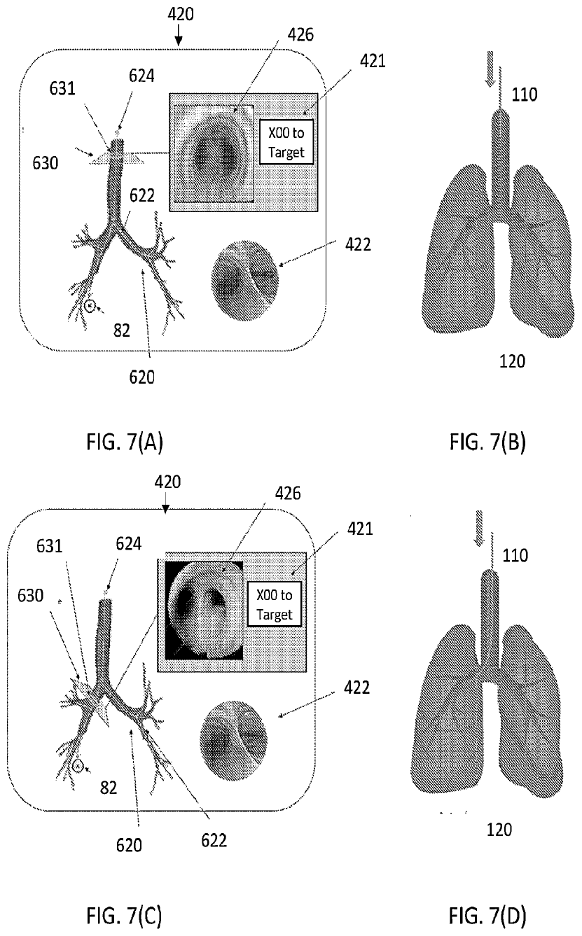

The guidance reference plane 630 can be updated to reflect only the insertion depth position of the catheter tip 104. In FIG. 7(A), the bendable medical device 110 is entering the airway, and the display screen 420 shows the guidance reference plane 630 at the corresponding to the insertion depth position of the catheter tip 104. For reference, the position in the lung 120 where the bendable medical device 110 is located is shown in FIG. 7(B). In FIG. 7(C), the bendable medical device 110 has navigated further into the lungs as compared to FIG. 7(A). This can be seen by the bendable medical device 110 position in the lung 120 in FIG. 7(D). Again, the corresponding the guidance reference plane 630 shown in FIG. 7(C) has moved down to that position shown. Note also that the guidance reference plane 630 is always perpendicular to the navigation path. Therefore, the position of guidance reference plane 630 can be determined as a plane perpendicular to the navigation path and including the current catheter tip 120 on its plane.

The guidance reference plan 630 gives the user a broader view “roadmap” than the live image endoscopic view 422 since more than just the instantaneous portion in the airway is shown.

All the possible airway direction options in the presentation of the airway structure 620 of the lung can be seen. Further, the progress along the navigation path 624 to the target 82 can easily be seen by the relative position of the guidance reference plane 630.

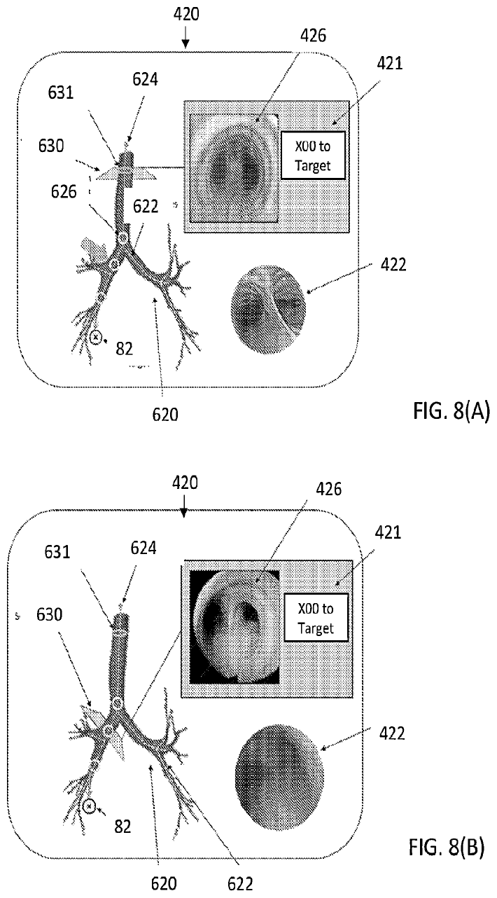

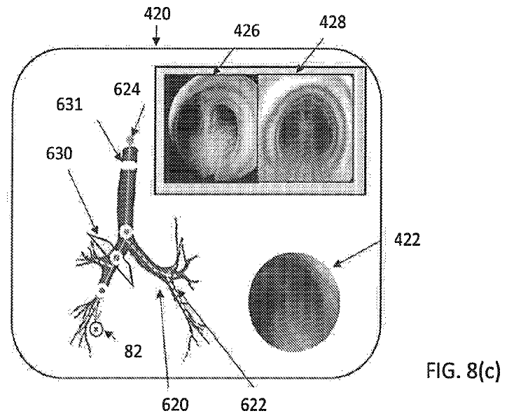

In other embodiments, the steerable medical system provides navigation points at the bifurcations 626 along the navigation path 624 (see FIG. 8(A)). Thus, if the user identifies a navigation point 626 (e.g. by clicking on the display using a mouse), the guidance reference plane moves to that position in the airway and the guidance virtual endoscope view 426 is adjusted such that it corresponds to the location of the selected navigation point 626. This is exemplified herein in FIG. 8(A). As the user clicks on the bifurcation point 626 marked by the green arrow in FIG. 8(A), the guidance reference plane 630 displayed moves to that location as shown by the guidance reference plane 630 in FIG. 8(B). The guidance virtual endoscope view 624 in FIG. 8(B) is similarly adjusted to correspond to the view at the navigation point selected by the user. However, note that while FIG. 8(A), which shows the actual location of the tip 104 of the bendable medical device 110 and the guidance reference plane 630 corresponding to the tip 104 of the bendable medical device 110, in display in FIG. 8(B), the tip position 104 and the corresponding live image endoscope view 422 remain unchanged since the bendable medical device was not moved. This allows for the user to preview what will be seen during navigation as the user progress down the planned path in the airway and at the same time have a reference for where in the airway the proposed navigational route is located. This also provides a means of review of what the user has just passed during navigation.

In FIG. 8(C), the guidance virtual endoscope view 426 is the same user-selected position as in FIG. 8(B). However, in this display 420, both a guidance virtual endoscope view 426 located at the selected bifurcation/navigation point 626, as well as a second virtual endoscope view 428 showing the position of the bronchoscope.

The guidance virtual endoscope view 426 coupled with the guidance reference plane 630 is orientation stable. Therefore the user can use structures shown in the oriented cross sectional bronchoscope view for orientation.

Displaying navigation points at the bifurcations 626 enable the user to preview (or review) anatomical structures along the navigation path. In some embodiments, there is an indicator (such as a marker or change in color) when the guidance virtual endoscope view 426 and/or the guidance reference plane 630 are shown during a preview or review step and is thus at a location that does not correspond to the position of the tip 104 of the bendable medical device 110.

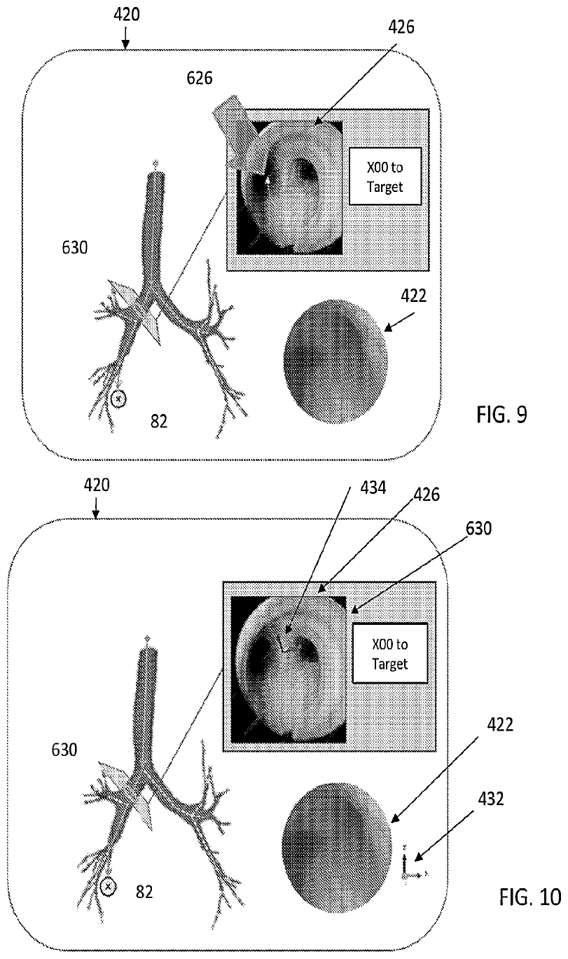

In some embodiments, navigation directions 624 can be overlaid on the virtual bronchoscope cross sectional view as well as on the patient's airway structure 620. This is shown in FIG. 9 as indicated by the arrow in the guidance virtual endoscope view 426.

As shown in FIG. 10, a XYZ indicator 432 is oriented in alignment to the tip of the catheter. This XYZ indicator 432 is updated in real time and reflects changes in the catheter tip direction and/or rotation. A different XYZ indicator 434 is overlaid on the virtual bronchoscope reference plane view 630. This XYZ indicator 434 reflects changes of the XYZ indicator 432, but displayed in the coordinate system of the virtual bronchoscope reference plane view 630. This allows the user to ascertain the orientation of the catheter tip while viewing the virtual bronchoscope reference view 630. This in turn, allows the user to make the necessary movement inputs to navigate in the desired direction in the virtual bronchoscope reference plane view 630.

The system 100 may be regulated, controlled, and/or directed by one or more processors in communication with the controller and optionally other components and/or subsystems of the overall system 1. The processor may operate based on instructions in a computer readable program stored in a non-transitory computer readable memory. The processor may be or include one or more of a CPU, MPU, GPU, ASIC, FPGA, DSP, and a general purpose computer. The processor may be a purpose built controller or may be a general purpose computing device that is adapted to be a controller. Examples of a non-transitory computer readable memory include but are not limited to RAM, ROM, CD, DVD, Blu-Ray, hard drive, networked attached storage (NAS), an intranet connected non-transitory computer readable storage device, and an internet connected non-transitory computer readable storage device.

Embodiment(s) of the present disclosure can be realized by computer system 400 or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer system may comprise one or more processors (e.g., central processing unit (CPU) 410, micro processing unit (MPU)) and may include a network of separate computers or separate processors to read out and execute the computer executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. For example, the present disclosure has been described above in terms of exemplary embodiments. However, there are many variations not specifically described to which the present disclosure could be applicable. For example, while the various embodiments are described with respect to an endoscope for use in medical procedures, the disclosure would be also applicable with respect to mechanical procedures of a borescope for use within various mechanical structures. Therefore, the scope of the following claims is to be accorded the broadest interpretation so as to encompass all such modifications and equivalent structures and functions.

1. A navigation system comprising:
a display device; and
a controller configured to display on the display device:
an image of a biological lumen imaged from the distal end of a bendable medical device that is in the biological lumen; and
a representation of an airway structure;
wherein the representation of the airway structure further comprises:
a navigation path through at least a portion of the airway structure; and
a guidance reference plane oriented perpendicular to the navigation path and located at an insertion depth of the distal end of the bendable medical device in the biological lumen.

2. The navigation system of claim 1, wherein the representation of the airway structure comprises:
a centerline, a target site, or both the centerline and the target site are shown on the representation of the airway structure.

3. The navigation system of claim 1, wherein the controller is further configured to display a guidance virtual endoscope view having an image center at the centerline on the guidance reference plane and a view orientation of the distal direction along the orientation of the guidance reference plane.

4. The navigation system of claim 3, wherein the controller is further configured to display a second virtual endoscope view having an image center at the distal end of the bendable medical device.

5. The navigation system of claim 1, wherein the controller is further configured to display on the display device navigation information comprising at least one of:
a navigation modality,
a distance from the bendable medical device to target site;
an insertion depth of the bendable medical device;
an information as to the position of a marker displayed on the airway structure; and
a warning(s) that the bendable medical device is reaching a threshold limit of force or
bending angle.

6. The navigation system of claim 1, wherein the controller is configured to initiate a corrective action when the bendable medical device is reaching a threshold limit of force or bending angle.

7. The navigation system of claim 1, wherein the controller is configured to update the display of the guidance reference plane as the bendable medical device is moved through the airway structure.

8. The navigation system of claim 1, wherein the guidance reference plane is displayed as having a three-dimensional perspective.

9. The navigation system of claim 1, wherein the representation of the airway structure is a model of a patient airway based on one or more computerized tomography (CT) scanner data and/or magnetic resonance imaging (MRI) scanner data.

10. The navigation system of claim 1, wherein the representation of the airway structure further includes one or more markers indicating one or more bifurcation points of the airway structure.

11. The navigation system of claim 1, wherein the guidance reference plane is instead located at a bifurcation point of the airway structure and is an overview guidance reference plane.

12. The navigation system of claim 11, wherein the bifurcation point of the airway structure is selected based on a user input, wherein the user input comprises selecting a markers indicating one or more bifurcation points of the airway structure located on the representation of the airway structure.

13. A medical system comprising:
a bendable medical device;
an actuator system for actuating the bendable medical device; and
the navigation system of claim 1.

14. The medical system of claim 13, wherein the controller is further configured to display a guidance virtual endoscope view having an image center at the centerline on the guidance reference plane and a view orientation of the distal direction along the orientation of the guidance reference plane.

15. A method for controlling a display, the method comprising:
acquiring an image of a biological lumen from the distal end of the bendable medical device;
obtaining a representation of an airway structure;
obtaining a target site;
generating a centerline of at least a portion of the airway structures;
generating a navigation path through at least a portion of the airway structure to the target site;
displaying, on a display device, the representation of an airway structure, wherein the a representation of an airway structure further comprises:
a navigation path through at least a portion of the airway structure; and
a guidance reference plane oriented perpendicular to the navigation path and located at an insertion depth of the distal end of the bendable medical device in the biological lumen.

16. The method of claim 15, further comprising displaying, on the display device, the image of a biological lumen.

17. The method of claim 15, wherein the representation of an airway structure is obtained from a pre-operative CT image.

18. The method of claim 15, wherein the target site is obtained from a user.
